(19) United States
(12) Patent Application Publication
(10) Pub. No.: US 2012/0046697 A1
(43) Pub. Date: Feb. 23, 2012

(54) Anchoring element and method

(75) Inventors: Zvi Laster, Poriya Elite (IL); Ole T. Jensen, Engelwood, CO (US); Meir Mamraev, Rishon Lezion (IL); Oved Gihon, Nes Ziona (IL)

(73) Assignee: CORTEX DENTAL IMPLANTS INDUSTRIES LIMITED

(21) Appl. No.: 13/137,265

(22) Filed: Aug. 2, 2011

Related U.S. Application Data
(60) Provisional application No. 61/401,697, filed on Aug. 18, 2010.

(51) Int. Cl. A61B 17/86

(52) U.S. Cl. 606/301

(57) ABSTRACT

An anchoring element for use in bone has an apical end and a distal end. The apical end may be embedded in bone while the distal end may be left exposed. An abutment surface is disposed toward the distal end. The anchoring element may include first, second, third and fourth surfaces that define the apical end. The fourth surface may be disposed between an apical end and a distal end of the first surface, may joined to the third surface along an outer circumference of the fourth surface and may be employed to shave, cut or route an upstanding core remaining in the bone.

### CROSS-REFERENCE TO RELATED APPLICATIONS

This application claims priority to U.S. Provisional Patent Application Ser. No. 61/401,697, filed Aug. 18, 2010, titled “Anchoring Element and Method”.

### FIELD OF THE DISCLOSURE

The present disclosure generally relates to anchoring elements designed to be used in a variety of dental, medical, and surgical procedures where it is desired to embed a mechanical attachment into living bone.

### BACKGROUND

Various different methods or techniques are known for securing dental prostheses with a patient's oral cavity. One illustrative example of a procedure that uses anchoring elements may be dental prostheses using embedded anchoring elements. However, the exemplary embodiments, and the description and drawings disclosing the same, should be interpreted by way of illustrative purposes only without limiting the scope and sprit of the present invention disclosure. Other types of procedures may well be considered as applicable for the utilization of the presently disclosed anchoring elements.

More particularly, dental anchoring elements may provide a desirable prosthesis for patients who are missing one or more natural teeth. A dental anchoring element may include an anchoring element that may be embedded into the jawbone and a prosthetic tooth that is attached to and supported by the anchoring element. The prosthetic tooth may be attached directly to the anchoring element or an abutment fixture may be attached to the anchoring element and support the prosthetic tooth in turn. An appropriate anchoring element will support bone growth that invades the anchoring element such that the anchoring element becomes integrated with the surrounding bone in a process termed osseointegration. However, other types of anchoring elements, designed to be embedded and/or integrated with living bone tissue, may also be included in the spirit and scope of the present disclosure.

An anchoring element for supporting a prosthetic tooth may be embedded in what is termed a two-stage procedure. In the first stage, the anchoring element is embedded into the jawbone and the surgical site is then closed. After a period of months the anchoring element will achieve osseointegration. The site of the anchoring element is then re-opened surgically to allow the attachment of a prosthetic tooth.

Other techniques may be employed to permit a dental anchoring element to be embedded in a one-stage procedure. In a one-stage procedure, the anchoring element is embedded and a prosthetic tooth is immediately fitted thereto. The immediately fitted prosthetic tooth may be an interim prosthesis that allows the soft tissue to properly heal and maintains the spacing and alignment of adjacent teeth during the period of osseointegration. A permanent prosthetic tooth may be fitted at a later date after at least some osseointegration has occurred, generally without requiring an additional surgical procedure.

To achieve successful osseointegration it is desirable that the anchoring element fit closely into the surrounding bone. It is also be desirable that the anchoring element does not move relative to the surrounding bone during the period of osseointegration. Where the anchoring element cannot be closely fitted to the surrounding bone, it may be necessary to use bone-grafting materials to fill the space between the anchoring element and the surrounding bone.

Molars are commonly missing teeth. However, many times, the use of an anchoring element to replace a molar may present some special difficulties, as molars may generally have multiple roots. The mandibular molars of the lower jaw generally have two roots. The maxillary molars of the upper jaw generally have three roots. In a fresh extraction site the void left by the molar roots presents a site that can be difficult to fit with an anchoring element. In addition, the bone in the molar region of the jaw may generally consist of a thin, hard layer of cortical bone surrounding a core of softer, spongy, cancellous bone. The cancellous bone may provide a lesser, or an even poorer support, for the anchoring element.

As molars may generally be relatively large teeth, it may be desirable to use an anchoring element having a relatively large diameter to fill the void following an extraction. However, the use of a wide anchoring element may require that a significant amount of bone be removed from the extraction site to accommodate the anchoring element. This may leave only a small amount of cortical bone available to support the anchoring element which may be embedded in predominantly cancellous bone. In particular, the use of a wide anchoring element may require the removal of a triangular mound-shaped mass of bone that is found between the roots and is known as the interradicular bone. Thus, it may prove difficult to place an anchoring element in a fresh molar extraction site with sufficient stability to allow the embed-ding of the dental anchoring element.

An option sometime practiced during the anchoring procedure of molars is called or referred to as “asymmetric loading”. In asymmetric loading, an anchoring element may be implanted in only one of the two (in the case of a mandibular molar extraction) or three (in the case of a maxillary molar extraction) root voids that exist post extraction. Such optional asymmetric loading may lead to reduced stability, which may lead to undesirable osseointegration and/or reduced strength and/or stability of the anchoring element.

Another circumstance that can present difficulties in placing an anchoring element within a patient's mouth may occur in cases where it is desired to place a dent-al prosthesis into a healed extraction site. When a molar is lost, the alveolar ridge that supports the teeth may be fairly rapidly re-absorbed. This may lead to a loss of height of the jawbone in the area of tooth loss. When an anchoring element is to be embedded into a healed extraction site, it may not be possible to insert the anchoring element to a desirably large depth.

In the lower jaw, the presence of the mandibular alveolar nerve in the lower jaw may limit the depth to which the anchoring element can be inserted. In the upper jaw, the maxillary sinus may likewise limit the depth to which the anchoring element can be inserted. These limiting anatomical features may require the use of a short anchoring element, the use of which for such cases may result in a lower rate of long-term success because of the reduced surface area available for osseointegration.

It would be desirable to have an anchoring element that can be used to place a dental anchoring element into the molar area both for fresh extraction sites and for healed sites, as well as an associated method therefor, which may facilitate the above. This may be attained with the subject matter in accordance with the following specification and claims.

### SUMMARY

In the following disclosure, aspects thereof are described and illustrated in con junction with systems and methods which are meant to be exemplary and illustrative, not limiting in scope. In various embodiments, one or more of the above-described issues and/or desirable effects have been addressed, while other aspects are directed to effect other advantages or improvements.

According to one aspect of the present disclosure, an anchoring element for use in bone is provided, the anchoring element having a distal end and an apical end.

Preferably, the anchoring element to which the present disclosure relates may comprise an anchoring element body having a longitudinal axis ξ defining an apical-to-distal direction and a threading-in direction τᵢ.

Furthermore, the anchoring element comprises a first surface having an apical end, a distal end, an annular second surface disposed toward the apical end of the first surface and joined to the first surface along an outer circumference of the second surface, a third surface concentric with the first surface and joined to the second surface along an inner circumference of the second surface, and a fourth surface interposed between the apical end and the distal end of the first surface and joined to the third surface along a distal end of the third surface and an outer circumference of the fourth surface, so that the fourth surface comprises at least one tub featuring an upstanding sidewall extending generally apically away from a tub floor so as to terminate in a generally apically-facing top surface portion, the upstanding sidewall meeting the top surface portion at an upper surface edge.

According to another aspect of the present disclosure, the first, second and third surfaces may be interrupted by at least one slot breaching the first, second and third surfaces and extending away from the apical end towards the fourth surface.

Potentially, the at least one slot may define at least one fluke extending circumferentially in the threading-in direction τi from a fluke leading face to a fluke trailing face.

Furthermore, the at least one fluke may extend generally away from the second surface towards the fourth surface.

Optionally, both the fluke leading face and the fluke trailing face may extend away from the second surface towards the fourth surface.

Moreover, the fluke leading face may define an outer leading edge as it meets the first surface and an inner leading edge as it meets the third surface, while the fluke trailing face may define an outer trailing edge as it meets the first surface and an inner trailing edge as it meets the third surface.

Furthermore, the fluke leading face may extend between the outer leading edge and the inner leading edge, while the fluke trailing face may extend between the outer trailing edge and the inner trailing edge.

Preferably, the at least one tub is defined by a generally apically facing tub floor bordered by the third surface and by an upstanding sidewall extending generally apically away therefrom so as to terminate in a generally apically-facing top surface portion.

Furthermore, the upstanding sidewall may meet the top surface portion at an upper surface edge.

Additionally, the at least one tub may communicate with, and open to, the first surface through the at least one slot.

According to a further aspect of the present disclosure, the fluke leading face may be angled at a leading angle λ relative to a tangent T_{λ} to the first surface at the outer leading edge, while optionally the fluke trailing face may be angled at a trailing angle θ to a tangent T_{θ} to the first surface at the outer trailing edge.

Preferably, either one or both of the leading angle λ, and the trailing angle θ may be constant, however, alternatively, either one or both of the leading angle λ and the trailing angle θ vary along an axial extent E of the slot.

Potentially, the leading angle λ, may be acute.

According to a further embodiment of the present disclosure, a normal v_{λ} to the fluke leading face at the outer leading edge may be directed generally inwardly to-wards the longitudinal axis ξ.

Preferably, the fluke leading face may be angled at a lead angle λ relative to a tangent T_{λ}, to the first surface at the inner leading edge.

Alternatively, a normal v_{λ} to the fluke leading face at the outer leading edge may be directed generally outwardly away from the longitudinal axis ξ.

Yet another aspect of the present disclosure comprises a method for placing an anchoring element into the oral cavity which is intended to receive an attachment to a bone, the method comprising providing a first surface of the anchoring element, the first surface having a distal end and an apical end, providing an annular second surface disposed toward the apical end of the first surface and joined to the first surface along an outer circumference of the second surface, providing a third surface concentric with the first surface and joined to the second surface along an inner circumference of the second surface, providing a fourth surface disposed between the apical end and the distal end of the first surface and joined to the third surface along an outer circumference of the fourth surface, and providing at least one tub featuring an upstanding sidewall extending apically away from a tub floor so as to terminate in a generally apically-facing top surface portion, the upstanding sidewall meets the top surface portion at an upper surface edge so that when placing the anchoring element into a bone by inserting the first surface into an outer wall of the hole being formed within the bone, the fourth surface may come into contact and/or engagement with an upper upstanding top surface of an upstanding core in the bone.

Preferably, as contact and/or engagement of the upper upstanding top surface is made by a top surface portion of the anchoring element, the upper surface edge may trim and/or root and/or cut away bone chips and/or tissue remnants, so as to prepare the upper upstanding top surface for abutment with a top surface portion.

Still further, the first, second and third surfaces may be interrupted by at least one slot which may breach the first, second and third surfaces and extend away from the apical end towards the fourth surface.

Optionally, a flute leading face may be defined by the at least one slot intersecting the first surface and the third surface and facing generally the threading-in direction τi.

Moreover, the flute leading face may define an acute leading edge at the meeting thereof with either the first surface or the third surface.

Furthermore, bone chips and/or tissue remnants, which may have been trimmed, cut and/or rooted by the upper surface edge may accumulate in the at least one tub to assist in enhancing osseointegration of the anchoring elements. Moreover, bone chips and/or tissue remnants which may have been cut and/or rooted and/or trimmed during implantation, may escape through the at least one slot, so as to re-duce the pressure upon healthy bone and/or to assist in osseointegration and to pre-vent turning after osseointegration.

In addition to the exemplary aspects and embodiments described above, further aspects and embodiments will become apparent by reference to the following detailed description and drawings.

### BRIEF DESCRIPTION OF EXEMPLARY DRAWINGS

Exemplary embodiments are illustrated in referenced drawing figures. It is intended that the embodiments and figures disclosed herein are to be considered illustrative rather than restrictive. It is emphasized that, according to common practice, the various features of the drawing are not to scale, but rather dimensions of various features are arbitrarily expanded or reduced for clarity.

Reference will now be made to the accompanying drawings, in which:

FIG. 1 is a perspective view of the apical end of a exemplary first exemplary anchoring element for use within bone that illustratively embodies the present disclosure;

FIG. 2 is a perspective view of the distal end of the first anchoring element shown in FIG. 1;

FIG. 3 is a cross-sectional view of the first anchoring element taken along the plane 3-3 shown in FIG. 1;

FIG. 4 is a perspective view of the apical end of a second exemplary anchoring element for use within bone in accordance with the teachings and principies of the present disclosure;

FIG. 5 is a perspective view of the distal end of the second anchoring element shown in FIG. 4;

FIG. 6 is a top plan view of the apical end of the second anchoring element shown in FIG. 5;

FIG. 7 is a perspective view of the apical end of a third exemplary anchoring element for use within bone that also illustrates the principles and teachings of the present disclosure;

FIG. 8 is a perspective view of the distal end of the third anchoring element shown in FIG. 7; and

FIG. 9 is a top plan view of the apical end of the third anchoring element shown in FIG. 7.

### DETAILED DESCRIPTION

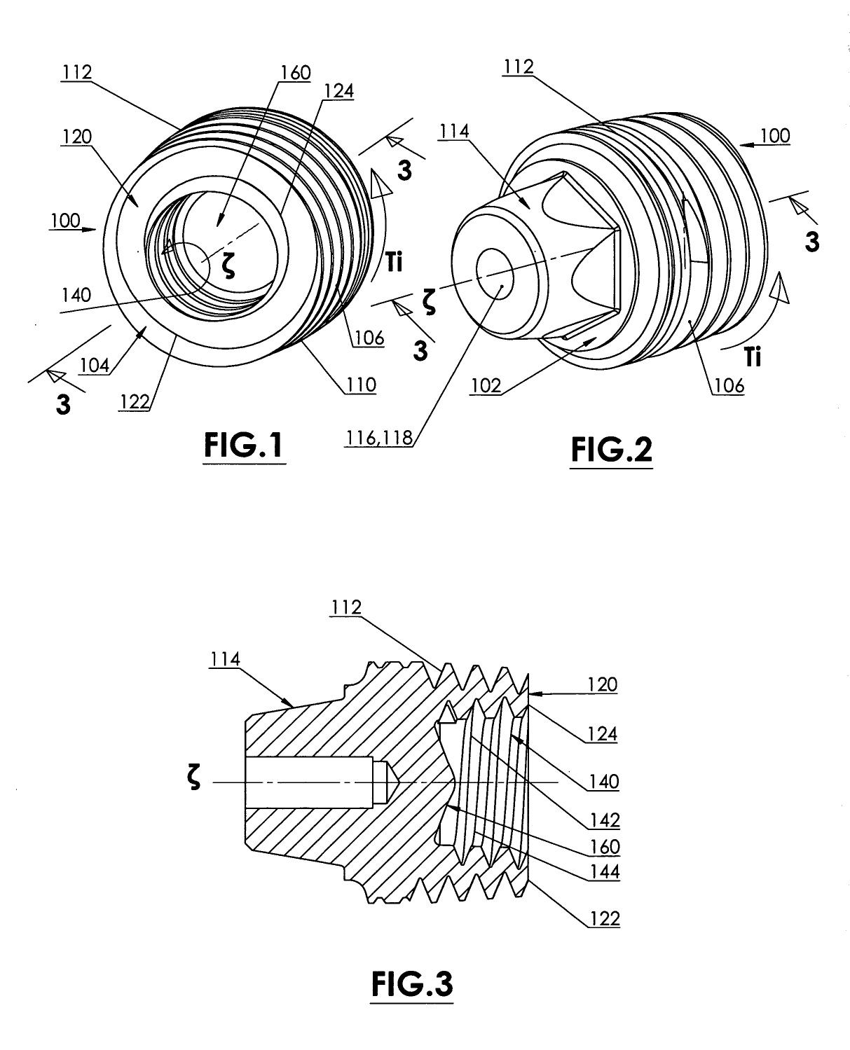

Attention is firstly drawn to FIGS. 1 to 3. For convenience of description, a first anchoring element 100 will be described with reference to a distal end 102 and an apical end 104. The apical end 104 is shown in FIG. 1 as the end to the lower left of the drawing and is the end that is embedded to the greatest depth into a bone (not shown). The distal end 102 is shown in FIG. 2 as the end to the lower left of the drawing, and is the end that may be exposed when the first anchoring element 100 is embedded within the bone (not shown), as well as the end to which an attachment (not shown) may be connected with the first anchoring element 100 either integrally therewith and/or removably therefrom. The first anchoring element 100 extends from the distal end 102 to the apical end 104 along a longitudinal axis ξ, defining a circumferential threading-in direction τᵢ.

The first anchoring element 100 may include a first surface 106 that comprises a generally cylindrical exterior surface of the anchoring element. The term “generally cylindrical” is used to describe a surface that is substantially rotationally symmetric about the longitudinal axis ξ. The first surface 106 may include a screw thread. It will be appreciated that a screw thread is not rotationally symmetric in the strictest sense, however a screw thread or any similar feature is intended to be included by the term “substantially rotationally symmetric.” The exterior surface of the first anchoring element 100 may include various features such as the aforementioned screw thread and other features such as shoulders, tapered portions, rings, ridges and the like (all not shown), all of which are intended to be included by the term “generally cylindrical”.

At least a portion of the first surface 106 of the first anchoring element 100 may include a first external thread 112 having a pitch. The first external thread 112 may be of any of a variety of forms known or discovered to be effective for embedding the anchoring element within a bone. Furthermore, the first external thread 112 may include one or more thread starts (not shown). The first external thread 112 may include self-threading (thread cutting) features or other features that may aid in embed-ding the first anchoring element 100 into bone, and/or that may promote osseointegration.

The first surface 106 may include a frustrated first conical portion 110 adjacent to the apical end 104 of the first anchoring element 100 such that a smaller circumference (not shown) of the first conical portion 110 is adjacent the apical end 104. The first conical portion 110 may facilitate placing the first anchoring element 100 into a hole (not shown) that may be prepared within the bone (not shown) or, alternatively, to receive the first anchoring element 100. Alternatively, the first conical portion 110 may facilitate insertion of the first anchoring element 100 without preparing a hole within the bone, so that the first anchoring element 100 may self-prepare a hole in which it is to be inserted. The first conical portion 110 may provide a somewhat smaller leading apical end that allows the first anchoring element 100 to initially engage the bone with minimal force and align the anchoring element with respect to the hole that has been prepared in the bone, or within the hole that the first anchoring element 100 self-prepares.

FIG. 2, in particular, may illustrate an exemplary abutment surface 114 that may be provided to receive a dental prosthesis. The exemplary abutment surface 114 may include wrenching surface or structure that provides a suitable surface to effectively be gripped by a tool for embedding the first anchoring element 100. Various known or discovered wrenching structures may be employed. The exemplary abutment surface 114 may further include a locating recess 116 and an optional internal anchoring thread 118 to receive devices (not shown) that may be coupled to the first anchoring element 100 by a screw (not shown) and/or other appropriate coupling means.

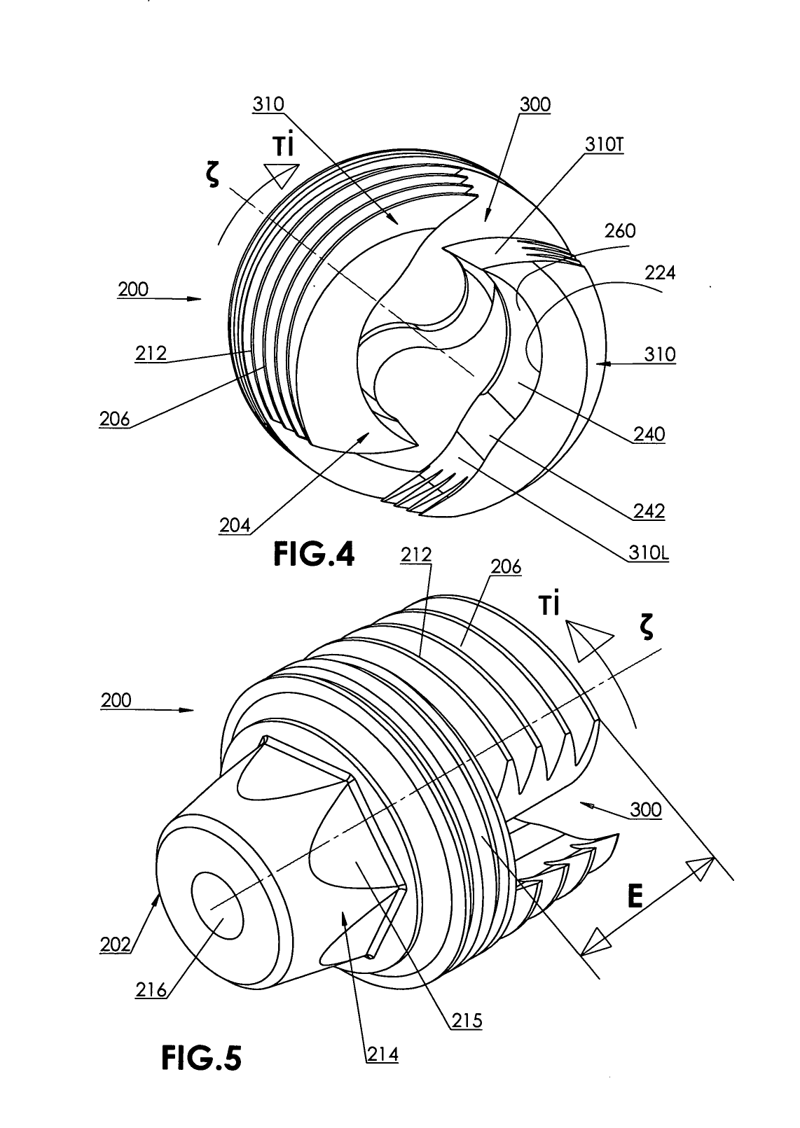

The first anchoring element 100 may include an annular second surface 120 disposed toward the apical end 104 of the first surface 106 and joined thereto along an outer circumference 122 of the second surface 120. The second surface 120 may engage the bottom (not shown) of the hole (not shown) that may have been prepared within the bone (not shown) and thereby contribute significantly to the stability of the first anchoring element 100 embedded therein. The second surface 120 may be substantially flat or may have a rounded shape or other configuration chosen to effectively cooperate with the bottom of the hole that has been prepared within the bone, or that the first anchoring element 100 self-prepared during insertion. As may be seen in FIGS. 1 and 3, the first anchoring element 100 may include a third surface 140 concentric with the first surface and joined to the second surface 120 along an inner circumference 124 of the second surface 120. The third surface 140 may extend away from the apical end 104 or from the second surface 120 generally distally, to an inner fourth surface 160.

At least a portion of the third surface 140 may include a third internal structure 142 which may be of any of a variety of forms known or discovered to be effective for embedding the anchoring element within the bone, such as screw threads and other structures such as shoulders, tapered portions, rings, ridges, corrugations and the like (only a screw thread is shown), all of which are intended to aid in embedding the first anchoring element 100 into the bone, or to promote osseointegration. The third internal structure 142 may include self-cutting, such as, but not limited to, self-threading (thread cutting) structures or other elements or components that may aid in embedding the first anchoring element 100 into the bone, or that promote osseointegration.

It is contemplated that the hole (not shown) within the bone (not shown) that is prepared to receive the first anchoring element 100, or the hole (not shown) which the first anchoring element 100 self-prepares during insertion, may leave a residual, upstanding core of bone (not shown) such that the third surface 140 will at least partially abut and/or engage the upstanding core so as to provide additional initial stability and/or an additional surface for osseointegration.

The third surface 140 may include a frusto-conical second portion 144 such that a larger circumference C of the second conical portion 140 is adjacent the apical end 104. The second frusto-conical portion 144 may facilitate the insertion of the first anchoring element 100 into that portion of the hole (not shown) that includes the up-standing core (not shown). The second frusto-conical portion 144 and/or the internal thread 142 formed thereon may thread into and/or compress the upstanding core (not shown) of the residual bone, as the first anchoring element 100 is being embedded, so as to further increase the initial stability, resist pulling, and/or enhance the bending resistance of the first anchoring element 100.

FIGS. 4 through 6 illustrate a second exemplary embodiment of an anchoring element 200 that also illustrates the principles and teachings of the present disclosure. The second anchoring element 200 may include many features that are similar to those of the first anchoring element 100 illustrated by FIGS. 1 through 3. Similar features for the second anchoring element 200 have been given reference numerals that are similar to the reference numerals used for first anchoring element 100 as in-creased by 100. For convenience in description, the second anchoring element 200 will be described with reference to a distal end 202 and an apical end 204. The apical end 204 is shown in FIG. 4 as the end to the lower right of the drawing and is the end that is embedded to the greatest depth in bone. The distal end 202 may be best seen in FIG. 5 as the end to the lower left of the drawing, and is the end that may be exposed when the second anchoring element 200 is embedded within the bone, as well as the end to which an attachment (not shown) may be connected to the anchoring element 200 either integrally therewith and/or removably therefrom. The second anchoring element 200 extends from the distal end 202 to the apical end 204 along the longitudinal axis ξ, defining a circumferential threading-in direction τᵢ.

The second anchoring element 200 includes a first surface 206 that is a generally cylindrical exterior surface of the anchoring element. Limitations related to the term “generally cylindrical” as used above also apply to the second anchoring element 200. At least a portion of the first surface 206 of the second anchoring element 200 includes a first external thread 212 having a pitch. The first external thread 212 may be of any of a variety of forms known or discovered to be effective for embedding the anchoring element within the bone. The first external thread 212 may include self-threading (thread cutting) structures or other elments or components that aid in the embedding of the second anchoring element 200 into the bone, or that promote osseointegration.

As may best seen in FIG. 5, the second anchoring element 200 may include an abutment surface 214 disposed toward the distal end of the first surface 206.

The abutment surface 214 may be of any of a variety of known or discovered forms that permit attachment to the second anchoring element 200. In this embodiment, the second anchoring element 200 includes an abutment surface 214 which is in the form of a frusto-conical pyramid having flats 215 formed on its flukes. The flats 215 formed upon the abutment surface 214 may provide wrenching surfaces. If the anchoring element is used to support a dental prosthesis, a prosthetic tooth may be directly coupled to the abutment surface 214 such as by a cement of any suitable type and/or other suitable adhesives. Alternatively, the abutment surface 214 may further include a locating recess 216 and an internal anchoring thread (not shown) to receive a device (not shown) that may be coupled to the second anchoring element 200 by a screw (not shown).

The second anchoring element 200 includes an annular second surface 220 disposed toward the apical end 204 of the first surface 206 and joined thereto along an outer circumference 222 of the second surface 220. The second surface 220 may en-gage the bottom (not shown) of a hole (not shown) that may have been prepared in the bone (not shown) and thereby contribute significantly to the stability of the second anchoring element 200 embedded therein. The second surface 220 may be substantially flat or may have a rounded shape or other configuration chosen to effectively cooperate with the bottom of the hole that has been prepared within the bone.

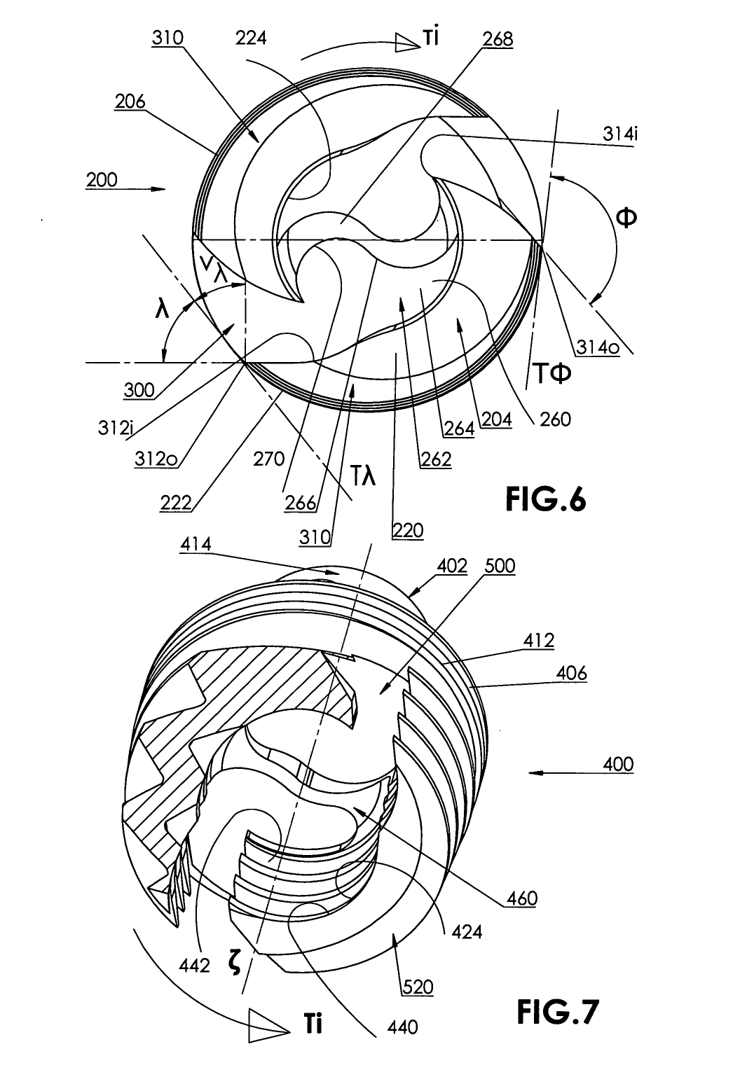

As may be seen in FIGS. 4 and 6, the second anchoring element 200 includes a third surface 240 generally concentric with the first surface and joined to the second surface 220 along an inner circumference 224 of the second surface 220. The third surface 240 may extend away from the apical end 204 or from the second surface 220 to an inner fourth surface 260. While the third surface 240 is shown as a smooth surface, at least a portion of the third surface 240 may include a third internal structure 242 which may be any one of a variety of forms known or discovered to be effective for embedding the anchoring element within the bone, such as screw threads and/or other elements or components such as, but not limited to, shoulders, tapered portions, rings, ridges, corrugations and the like (not shown), all of which are intended to aid in the embedding of the second anchoring element 200 into bone, or to promote osseointegration. The third internal structure 242 may include self-cutting structures, such as, but not limited to, selfthreading (thread cutting) structures or other elements or components that may aid in the embedding of the anchoring element 200 into the bone, or that promote osseointegration.

It is contemplated that the hole in the bone that is prepared to receive the second anchoring element 200 may leave a residual, upstanding core of bone (not shown) such that the third surface 240 will at least partially engage the upstanding core so as to provide additional initial stability and an additional surface for osseointegration.

Still referring to FIGS. 4 to 6, the first, second and third surfaces 206, 220, 240 may be interrupted by at least one slot 300 which breaches the first, second and third surfaces 206, 220, 240 and which extends away from the apical end 202 towards the fourth surface 260. The at least one slot 300 defines an at least one fluke 310 ex-tending circumferentially in the threading-in direction τᵢ from a fluke leading face 310L to a fluke trailing face 310T. The at least one fluke 310 extends generally away from the second surface 220 towards the fourth surface 260. Both the fluke leading face 310L and the fluke trailing face 310T extend away from the second surface 220 towards the fourth surface 260.

The fluke leading face 310L defines an outer leading edge 312o as it meets the first surface 206, and an inner leading edge 312i as it meets the second surface 220, so that the fluke leading face 310L extends between the outer leading edge 312o and the inner leading edge 312i. Similarly, the fluke trailing face 310T defines an outer trailing edge 314o as it meets the first surface 206 and an inner trailing edge 314i as it meets the second surface 220, so that the fluke trailing face 310T extends between the outer trailing edge 314o and the inner trailing edge 314i.

The fluke leading face 310L may be angled at a leading angle λ relative to a tangent T_{λ} to the first surface 206 at the outer leading edge 312o, while the fluke trailing face 310T may be angled at a trailing angle θ to a tangent T_{θ} to the first surface 206 at the outer trailing edge 314o. Both the leading angle λ and the trailing angle θ may be constant, or may vary along the axial extent E of the slot 310. Preferably, the leading angle λ may be acute, while the trailing angle θ may be obtuse. A normal v_{λ} to the fluke leading face 310L at the outer leading edge 312o may be directed generally radially inwardly towards the longitudinal axis ξ. Thus, in the presently discussed embodiment, the outer leading edge 312o may be referred to as an acute leading edge.

Directing attention now to FIG. 4 and, particularly to FIG. 6, the fourth surface 260 may include at least one tub 262. The at least one tub 262 may be defined by a generally apically facing tub floor 264. The tub floor 264 may be bordered by the third surface 240 and by an upstanding sidewall 266 which may extend generally apically away from the tub floor 264 so as to terminate in a generally apically-facing top surface portion 268. The upstanding sidewall 266 meets the top surface portion 268 at an upper surface edge 270. The at least one tub 262 may communicate with, and open to, the first surface 206 through the at least one slot 300.

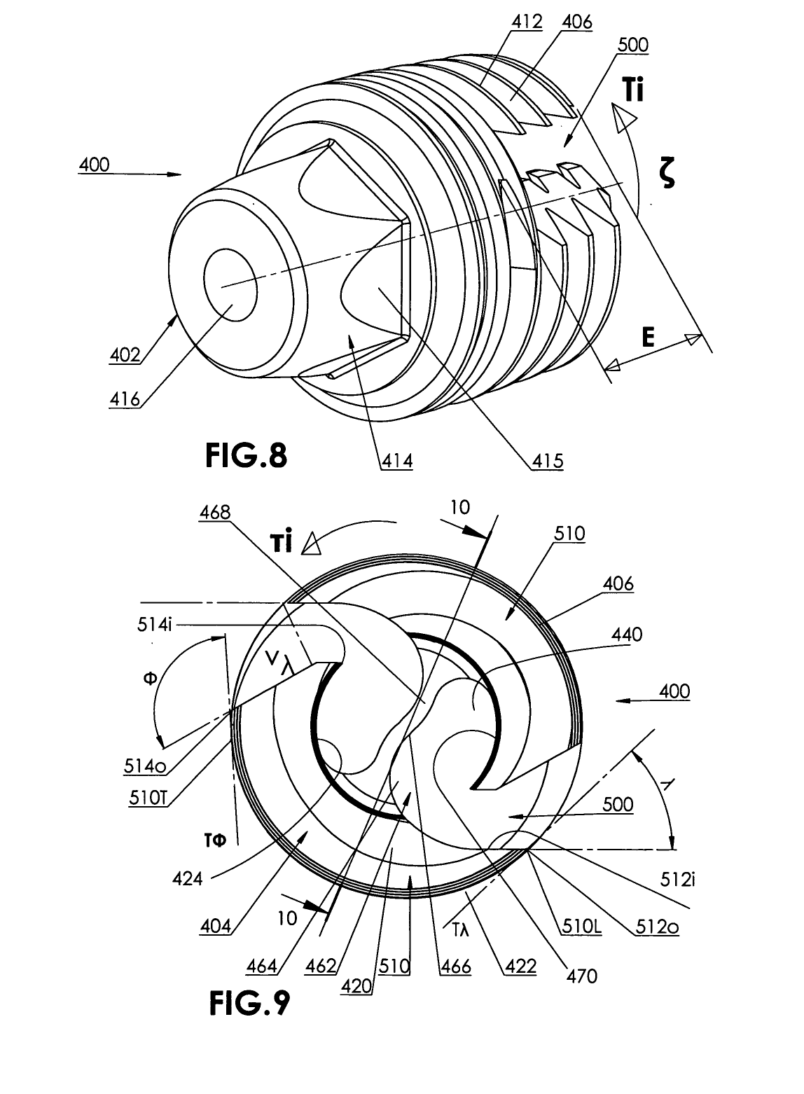

FIGS. 7 through 9 schematically illustrate a third exemplary embodiment of an anchoring element 400 that likewise illustrates the principles and teachings of the present disclosure. The third anchoring element 400 includes many features that are similar to those of the first anchoring element 100 illustrated by FIGS. 1 through 3 and to those of the second anchoring element 200 illustrated be FIGS. 4 through 6. Similar features for the third anchoring element 400 have been given reference numerals that are similar to the reference numerals used for the first anchoring element 100 increased by 300. For convenience in description, the third anchoring element 400 will be described with reference to a distal end 402 and an apical end 404. The apical end 404 is shown in FIG. 7 as the end to the lower left of the drawing and is the end that is embedded to the greatest depth within the bone. The distal end 402 may be best seen in FIG. 8 as the end to the lower left of the drawing, and is the end that may be exposed when the third anchoring element 400 is embedded within the bone, as well as the end to which an attachment (not shown) may be connected to the third anchoring element 400 either integrally therewith and/or removably therefrom. The third anchoring element 400 extends from the distal end 402 to the apical end 404 along the longitudinal axis ξ, defining a circumferential threading-in direction τᵢ.

The third anchoring element 400 includes a first surface 406 that is a generally cylindrical exterior surface of the anchoring element. Limitations related to the term “generally cylindrical” as have been used above also apply to the third anchoring element 400. At least a portion of the first surface 406 of the third anchoring element 400 may include a first external thread 412 having a pitch. The first external thread 412 may be of any one of a variety of forms known or discovered to be effective for embedding the anchoring element within the bone. The first external thread 412 may include self-threading (thread cutting) structures or other elements or components that aid in the embedding of the third anchoring element 400 into bone, or that promote osseointegration.

As may be best seen in FIG. 8, the third anchoring element 400 may include an abutment surface 414 disposed toward the distal end of the first surface 406.

The abutment surface 414 may be of an_{y} one of a variety of known or discovered forms that permit attachment to the third anchoring element 400. The third anchoring element 400 includes an annular second surface 420 disposed toward the apical end 404 of the first surface 406 and joined thereto along an outer circumference 422 of the second surface 420. As may be seen in FIGS. 7 and 9, the third anchoring element 400 includes a third surface 440 concentric with the first surface 406 and joined to the second surface 420 along an inner circumference 424 of the second surface 420. The third surface 440 may extend away from the apical end 404 or from the second surface 420 towards an inner fourth surface 460. While in FIG. 7 it may appear that the third surface 440 may be provided with corrugations (or wavy structure), at least a portion of the third surface 440 may include a third internal feature 442 which may be of any one of a variety of forms known or discovered to be effective for embedding the anchoring element 400 into bone, such as screw threads and/or other structures such as, but not limited to, shoulders, tapered portions, rings, ridges, corrugations and the like (not shown), all of which are intended to aid in the embedding of the third anchoring element 400 into bone, or to promote osseointegration.

It is contemplated that the hole in the bone that is prepared to receive the third anchoring element 400 may leave a residual, upstanding core of bone (not shown) such that the third surface 440 will at least partially engage the upstanding core to provide additional initial stability and an additional surface for osseointegration.

Referring again to FIGS. 7 to 9, the first, second and third surfaces 406, 420, 440 may be interrupted by at least one slot 500 breaching the first, second and third surfaces 406, 420, 440 and extending away from the apical end 402 towards the fourth surface 460. The at least one slot 500 defines an at least one fluke 510 which extends circumferentially in the threading-in direction τᵢ from a fluke leading face 510L to a fluke trailing face 510T. The at least one fluke 510 extends generally away from the second surface 420 towards the fourth surface 460. Both the fluke leading face 510L and the fluke trailing face 510T extend away from the second surface 420 towards the fourth surface 460.

The fluke leading face 510L defines an outer leading edge 512o as it meets the first surface 406 and an inner leading edge 512i as it meets the third surface 420, so that the fluke leading face 510L extends between the outer leading edge 512o and the inner leading edge 512i. Similarly, the fluke trailing face 510T defines an outer trailing edge 514o as it meets the first surface 406 and an inner trailing edge 514i as it meets the third surface 420, so that the fluke trailing face 510T extends between the outer trailing edge 514o and the inner trailing edge 514i.

The fluke leading face 510L may be angled at a leading angle ξ relative to a tangent T_{λ} to the third surface 420 at the inner leading edge 512i, while the fluke trailing face 510T may be angled at a trailing angle θ to a tangent T_{θ} to the third surface 420 at the inner trailing edge 514i. Both the leading angle and the trailing angle θ may be constant, or may vary along the axial extent E of the slot 510. Preferably, the leading angle λ may be acute, while the trailing angle θ may be obtuse. A normal v_{λ} to the fluke leading face 510L at the outer leading edge 512o may be directed generally radially in a direction away from the longitudinal axis ξ. Thus, in the presently discussed embodiment, the inner leading edge 512i may be referred to as an acute leading edge.

Directing attention now to FIG. 7 and also particularly to FIG. 9, the fourth surface 460 may include at least one tub 462. The at least one tub 462 may be defined by a generally apically facing tub floor 464. The tub floor 464 may be bordered by the third surface 440 and by an upstanding sidewall 466 which may extend generally apically away from the tub floor 464 so as to terminate in a generally apically-facing top surface portion 468. The upstanding sidewall 466 meets the top surface portion 468 at an upper surface edge 470. The at least one tub 462 may communicate with, and open to, the first surface 406 through the at least one slot 500.

Returning now to the present disclosure in general, during the anchoring operation or procedure, it is contemplated that the inner fourth surfaces 160, 260, 420 of the first, second, and third anchoring elements 100, 200, 400 (which, for convenience reasons, will be collectively referred to herein after as “anchoring elements”) may come into contact with an upper upstanding top surface (not shown) of the upstanding core (not shown). Initially, contact and/or engagement of the upper upstanding top surface is made by the top surface portions 168, 268, 468 of the anchoring elements. As such initial contact is made, the upper surface edges 170, 270, 470 may trim and/or root and/or cut away bone chips and/or tissue remnants (not shown) so as to prepare the upper upstanding top surfaces for abutment with the top surface portions 168, 268, 468.

Bone chips and/or tissue remnants, which may be trimmed, cut and/or rooted by the upper surface edges 170, 270, 470, may accumulate in the at least one tubs 262, 462 to assist in enhancing osseointegration of the anchoring elements. It is further contemplated that following the embedding of the anchoring elements in the bone, the cut, rooted and/or trimmed upstanding core may provide better support, enhanced stability, and/or may provide better resistance to any bending of the anchoring elements embedded within the bone.

Optionally, the normal v_{λ} to the fluke leading face 310L at the outer leading edge 312o of the second anchoring element 200, which may be directed generally radially inwardly towards the longitudinal axis ξ, may define an acute external cutting edge that may assist in the trimming and/or rooting surrounding the bone at an anchoring site. Alternatively, the normal v_{λ} to the fluke leading face 510L at the inner leading edge 512i of the third anchoring element 400, which may be directed generally radially outwardly away from the longitudinal axis ξ, may define an acute internal cutting edge that may assist in the trimming and/or rooting the upstanding core at the anchoring site.

It is further contemplated that, with the second anchoring element 200 and/or with the third anchoring element 300, during anchoring, little or no predrilling, particularly with trephine drills, may be required, as the acute lead angle λ of the acute leading edge 312o, 512i of the least one flukes 310, 510 for placing an anchoring element intended to receive an attachment to the bone may cut through any bone that is left between voids remaining after extraction of the molar roots. Moreover, it is further contemplated that bone chips and/or tissue remnants that were cut and/or rooted and/or trimmed during implantation, may escape through the slots 300, 500, to reduce pressure on healthy bone, as well as potentially to assist in the osseointegration and to prevent any turning or rotation after osseointegration.

All directional references (such as, but not limited to, upper, lower, upward, downward, left, right, leftward, rightward, top, bottom, above, below, vertical, horizontal, clockwise, and counterclockwise, tangential, axial and/or radial, or any other directional and/or similar references) are only used for identification purposes to aid the reader's understanding of the embodiments of the present disclosure, and may not create any limitations, particularly as to the position, orientation, or use unless specifically set forth in the claims. Similarly, joinder references (such as, but not limited to, attached, coupled, connected, and the like) are to be construed broadly and may include intermediate members between a connection of elements and relative movement between elements. As such, joinder references may not necessarily infer that two elements are directly connected and in fixed relation to each other.

In some instances, components are described with reference to “ends” having a particular characteristic and/or being connected with another part. However, those skilled in the art will recognize that the present disclosure is not limited to components which terminate immediately beyond their points of connection with other parts. Thus, the term “end” should be interpreted broadly, in a manner that includes areas adjacent, rearward, forward of, or otherwise near the terminus of a particular element, link, component, part, member or the like.

Additionally, all numerical terms, such as, but not limited to, “first”, “second”, “third”, or any other ordinary and/or numerical terms, should also be taken only as identifiers, to assist the reader's understanding of the various embodiments, variations and/or modifications of the present disclosure, and may not create any limitations, particularly as to the order, or preference, of any embodiment, variation and/or modification relative to, or over, another embodiment, variation and/or modification.

Similarly, adjectives such as, but not limited to, “articulated”, “modified”, or similar terms, should be construed broadly, and only as nominal, and may not create any limitations, particularly as to the description, operation, or use unless specifically set forth in the claims.

While the entire discussion relates to a seated position as a first position, and to a prone position as a second position, the opposite may equally apply, that is, the patient may be initially positioned in the prone position, and transferred passively to the seated position. Additionally, the patient may be positioned on the therapeutic apparatus in any interim position, and transferred substantially passively to any other position, either any interim position between the seated and prone positions, or the first and second positions themselves.

In methodologies directly or indirectly set forth herein, various steps and operations are described in one possible order of operation, but those skilled in the art will recognize that steps and operations may be rearranged, replaced, or eliminated without necessarily departing from the spirit and scope of the present disclosure as set forth in the claims. It is intended that all matter contained in the above description or shown in the accompanying drawings shall be interpreted as illustrative only and not limiting. Changes in detail or structure may be made without departing from the spirit of the present disclosure as defined in the appended claims.

While certain exemplary embodiments have been described and shown in the accompanying drawings, it is to be understood that such embodiments are merely illustrative of and not restrictive on the broad present disclosure, and that this present disclosure not be limited to the specific constructions and arrangements shown and described, since various other modifications and/or adaptations may occur to those of ordinary skill in the art. It is to be understood that individual features shown or described for one embodiment may be combined with individual features shown or described for another embodiment. It is also to be understood some features are shown or described to illustrate the use of the present disclosure in the context of functional elements and such features may be omitted within the scope of the present disclosure and without departing from the spirit of the present disclosure as defined in the appended claims.

While certain exemplary aspects and/or embodiments have been broadly described and/or schematically illustrated in the accompanying drawings, it is to be understood that such aspects and/or embodiments are merely illustrative of, and not restrictive on, the broad present disclosure. Furthermore, those of skill in the art may recognize that the present disclosure may not be limited to the specific constructions and arrangements shown and described, since various other modifications, permutations, additions and sub-combinations may occur to those ordinarily skilled in the art, without detracting from the spirit and scope of the present disclosure. It is to be understood that individual features shown or described for one embodiment may be combined with individual features shown or described for another embodiment. Furthermore, it is to be understood some features may have been shown or described to illustrate the use of the present disclosure in the context of functional anchoring elements and such features may be omitted within the,spirit and scope of the present disclosure.

1. An anchoring element for use in bone, the anchoring element comprising:
a first surface having an apical end and a distal end;
an annular second surface disposed toward the apical end of the first surface and joined to the first surface along an outer circumference of the second surface;
a third surface concentric with the first surface and joined to the second surface along an inner circumference of the second surface; and
a fourth surface disposed between the apical end and the distal end of the first surface and joined to the third surface along a distal end of the third surface and an outer circumference of the fourth surface, wherein
the fourth surface comprises at least one tub featuring an upstanding sidewall extending generally apically away from a tub floor to terminate in a generally apically-facing top surface portion, the upstanding sidewall meeting the top surface portion at an upper surface edge.

2. The anchoring element for use in bone of claim 1, wherein the first, second and third surfaces are interrupted by at least one slot breaching the first, second and third surfaces and extending away from the apical end towards the fourth surface.

3. The anchoring element for use in bone of claim 2, wherein the at least one slot defines an at least one fluke extending circumferentially in the threading-in direction τᵢ from a fluke leading face to a fluke trailing face.

4. The anchoring element for use in bone of claim 3, wherein the at least one fluke extends generally away from the second surface towards the fourth surface.

5. The anchoring element for use in bone of claim 4, wherein both the fluke leading face and the fluke trailing face extend away from the second surface towards the fourth surface.

6. The anchoring element for use in bone of claim 5, wherein the fluke leading face defines an outer leading edge as it meets the first surface and an inner leading edge as it meets the third surface, while the fluke trailing face defines an outer trailing edge as it meets the first surface and an inner trailing edge as it meets the third surface.

7. The anchoring element for use in bone of claim 6, wherein the fluke leading face extends between the outer leading edge and the inner leading edge, while the fluke trailing face extends between the outer trailing edge and the inner trailing edge.

8. The anchoring element for use in bone of claim 3, wherein the at least one tub is defined by a generally apically facing tub floor bordered by the third surface and by an upstanding sidewall extending generally apically away therefrom to terminate in a generally apically-facing top surface portion.

9. The anchoring element for use in bone of claim 8, wherein the upstanding sidewall meets the top surface portion at an upper surface edge.

10. The anchoring element for use in bone of claim 9, wherein the at least one tub communicates with, and open to, the first surface through the at least one slot.

11. The anchoring element for use in bone of claim 7, wherein the fluke leading face is angled at a lead angle λ relative to a tangent T_{λ} to the first surface at the outer leading edge.

12. The anchoring element for use in bone of claim 11, wherein the fluke trailing face is angled at a trail angle θ to a tangent T_{θ} to the first surface at the outer trailing edge.

13. The anchoring element for use in bone of claim 12, wherein the either or both of the lead angle λ and the trail angle θ are constant.

14. The anchoring element for use in bone of claim 12, wherein either or both of the lead angle λ and the trail angle θ vary along an axial extent E of the slot.

15. The anchoring element for use in bone of claim 11, wherein the lead angle λ is be acute.

16. The anchoring element for use in bone of claim 11, wherein a normal v_{λ} to the fluke leading face at the outer leading edge is directed generally inwardly towards the longitudinal axis ξ.

17. The anchoring element for use in bone of claim 7, wherein the fluke leading face is angled at a lead angle A relative to a tangent T_{λ} to the first surface at the inner leading edge.

18. The anchoring element for use in bone of claim 11, wherein a normal v_{λ} to the fluke leading face at the outer leading edge is directed generally outwardly away from the longitudinal axis τ.

19. A method for placing an anchoring element, intended to receive an attachment, into a bone, the method comprising the steps of:
providing a first surface of the anchoring element, the first surface having a distal end and a distal end;
providing an annular second surface disposed toward the apical end of the first surface and joined to the first surface along an outer circumference of the second surface;
providing a third surface concentric with the first surface and joined to the second surface along an inner circumference of the second surface;
providing a fourth surface disposed between the apical end and the distal end of the first surface and joined to the third surface along an outer circumference of the fourth surface, and
providing at least one tub featuring an upstanding sidewall extending apically away from a tub floor to terminate in a generally apically-facing top surface portion, the upstanding sidewall meets the top surface portion at an upper surface edge; wherein
placing the anchoring element into a bone by inserting the first surface into an outer wall of hole being formed in the bone until the fourth surface comes into contact and/or engagement with an upper upstanding top surface of an upstanding core in the bone.

20. The method for placing an anchoring element intended to receive an attachment in a bone of claim 19, wherein as contact and/or engagement of the upper upstanding top surface is made by an top surface portion of the anchoring element, the upper surface edge trim and/or route and/or cut away bone chips and/or tissue remnants, to prepare the upper upstanding top surface for abutment with a top surface portion.

21. The method for placing an anchoring element intended to receive an attachment in a bone of claim 19, wherein the first, second and third surfaces are interrupted by at least one slot breaching the first, second and third surfaces and extending away from the apical end towards the fourth surface.

22. The method for placing an anchoring element intended to receive an attachment in a bone of claim 21, wherein a flute leading face is defined by the at least one slot intersecting the first surface and the third surface and facing generally the threading-in Direction τᵢ.

23. The method for placing an anchoring element intended to receive an attachment in a bone of claim 22, wherein the flute leading face defines an acute leading edge at the meeting thereof either with the first surface or the third surface.

24. The method for placing an anchoring element intended to receive an attachment in a bone of claim 20, wherein bone chips and/or tissue remnants, trimmed, cut and/or routed by the upper surface edge are accumulate in the at least one tub to assist in enhancing osseointegration of the anchoring elements

25. The method for placing an anchoring element intended to receive an attachment in a bone of claim 24, wherein bone chips and/or tissue remnants that were cut and/or routed and/or trimmed during implantation escape through the at least one slot, to reduce pressure on healthy bone and/or to assist in osseointegration and to prevent turning after osseointegration.
